(19) United States
(12) Patent Application Publication
(10) Pub. No.: US 2026/0158060 A1
(43) Pub. Date: Jun. 11, 2026

(54) IRON NANOCLUSTERS, METHODS FOR OBTAINING SAME AND USES THEREOF FOR COMBATTING IRON DEFICIENCIES

(71) Applicants: Université de Lorraine, NANCY (FR); CENTRE HOSPITALIER REGIONAL UNIVERSITAIRE DE NANCY, NANCY (FR); INSTITUT NATIONAL DE LA SANTÉ ET DE LA RECHERCHE MÉDICALE, PARIS (FR)

(72) Inventors: Ariane BOUDIER, SAINT-MAX (FR); Igor CLAROT, EINVAUX (FR); François FEILLET, DOMBASLE SUR MEURTHE (FR)

(73) Assignees: Université de Lorraine, NANCY (FR); CENTRE HOSPITALIER REGIONAL UNIVERSITAIRE DE NANCY, NANCY (FR); INSTITUT NATIONAL DE LA SANTÉ ET DE LA RECHERCHE MÉDICALE, PARIS (FR)

(21) Appl. No.: 19/127,937

(22) PCT Filed: Nov. 9, 2023
(86) PCT No.: PCT/EP2023/081345

§ 371 Date: May 7, 2025

(30) Foreign Application Priority Data
Nov. 10, 2022 (EP) 22315281.0

(51) Int. Cl. A61K 33/26; A61K 9/51; A61K 47/12; A61K 47/22

(52) U.S. Cl. CPC A61K 33/26; A61K 9/5115; A61K 9/5192; A61K 47/12; A61K 47/22

(57) ABSTRACT

Iron nanoclusters that: are covered on their surface with a mixed layer comprising histidine (His), acetate ions (Ac), and ascorbate ions (Asc), have a spherical shape, have a hydrodynamic diameter ranging from 0.6 to 2.0 nm, have metal core diameter ranging from 0.5 to 1.5 nm, exhibit a stability duration ranging from 5 to 20 weeks in liquid form stored at 4° C., exhibit a stability of at least 12 months in dry form stored at 4° C. under nitrogen, exhibit spectrophotometric properties, with a shoulder in the UV-Visible spectrum at 300±15 nm and a fluorescence spectrum with excitation wavelengths of 364±15 nm and emission wavelengths of 415±15 nm. Also, a method of preparing the iron nanoclusters, and their uses for combating iron deficiencies.

### FIELD

The present invention relates to the field of chemistry, and more particularly to pharmaceutical chemistry. The invention concerns iron nanoclusters, methods for obtaining them, and their uses for combating iron deficiencies, more particularly their uses in the prevention and/or treatment of diseases that cause an iron deficiency.

### BACKGROUND

Iron deficiency, also known as hypoferremia and low iron levels, refers to a lack of iron in the body. Iron is a metal essential to the body's functioning and is derived solely from food. Iron deficiency is one of the most widespread mineral deficiencies in the world: more than 1.5 billion people suffer from iron deficiency worldwide.

Iron is present in all cells of the human body and is responsible for many vital functions, including oxygen transport via its presence in hemoglobin. Iron deficiency can interfere with these vital functions, the first sign being microcytic anemia, and in extreme cases can lead to death.

The majority of iron in the body (70%) is found in heme form, i.e., associated with hemoglobin in blood (65%) or myoglobin in muscles (5%). The remaining iron is found in non-heme form (ferritin, transferrin, etc.).

Iron absorption is a finely regulated mechanism. When the body's iron stores decrease, the iron absorption rate increases; conversely, when iron stores are high, absorption decreases, preventing excessive iron accumulation in the body (iron accumulation leads to iron overload). Diet provides an average of 10 to 15 mg of iron per day. Only 1 to 2 mg is absorbed, in the upper part of the small intestine. Iron absorption depends on its type, the quality of the meal, and the state of an individual's stores. 15 to 25% of heme iron is absorbed, compared to 2 to 20% for non-heme iron.

Iron deficiency can have several causes: increased need in an individual, decreased intake, malabsorption, chronic bleeding, and various diseases.

Iron deficiency is generally treated with oral iron preparations (tablets, capsules, powder, drops, syrup, etc.). When taken orally, the preparation reaches the stomach, then the iron is absorbed by the intestinal mucosa and thus enters the bloodstream.

However, oral iron supplementation can have several disadvantages, such as poor tolerance by the disgestive system. Those treated may complain of stomachaches, because some preparations release iron directly once the stomach is reached. Since the intestine can only absorb a limited amount of iron (maximum 20 to 25% for heme iron), a relatively large portion of the ingested iron is excreted. Thus, oral iron administration leads to frequent adverse effects, namely nausea, diarrhea or constipation, and black stools; more rarely, abdominal pain, a metallic taste in the mouth, or a blackish discoloration of the teeth which disappears when treatment is stopped.

Oral iron intake also requires considering the potential interactions with medications and foods. Indeed, certain medications and foods can bind oral iron in the digestive tract to form non-absorbable complexes, which significantly reduces its absorption.

Furthermore, oral iron preparations often cannot be used in certain types of patients, for example those with impaired iron absorption, particularly due to chronic inflammatory bowel disease. Also, oral iron preparations may simply not be effective in some cases, meaning that iron supplementation does not improve the individual's iron deficiency, in particular because the iron does not cross the intestinal barrier.

Thus, when it is not/no longer possible to administer oral iron preparations for the reasons mentioned above, or because there is a clinical need to administer iron rapidly, or because the recommended iron dosage in a patient is higher than the digestive tract's capacity to absorb iron, then intravenous iron may be administered, meaning that the iron is administered directly into the bloodstream via an intravenous infusion.

The advantage of the intravenous route is that the iron enters the bloodstream directly, and from there reaches the entire body. However, intravenous administration carries potential side effects such as hypersensitivity reactions, hypophosphatemic osteomalacia, impaired liver or kidney function, infection, extravasation during the infusion, etc., which make is essential to administer the iron in a hospital setting. This therefore requires hospitalization with the structural requirements inherent to that type of care (available beds, medical and nursing staff). Furthermore, the iatrogenic risks of this type of infusion are not zero.

In view of these difficulties, there is currently still a need to find an alternative to intravenous or oral iron treatments.

The present invention proposes novel iron nanoclusters for combating all types of iron deficiency, said nanoclusters advantageously being administered orally, without the above disadvantages.

Iron nanoclusters for combating iron deficiency have already been proposed in the literature. For example, patent US 2016/0022733 describes iron oxide nanocomposites capped with folic acid, nicotinic acid, and ascorbic acid, for use in treating anemia, these nanocomposites being intended for oral administration. Patent US 2016/008292 describes iron oxide nanoparticles coated with biocompatible polymers containing polyethylene glycol and silane groups, said groups being covalently bound via a linker. These nanoparticles are proposed for the parenteral treatment of anemia.

However, the inventors are credited with developing novel iron nanoclusters with characteristics that are particularly suitable for combating iron deficiency. The inventors are also credited with developing an original method for synthesizing the iron nanoclusters.

### SUMMARY

The present invention relates to iron nanoclusters which have the following characteristics:
they are covered on their surface with a mixed layer comprising histidine (His), acetate ions (Ac), and ascorbate ions (Asc),
they have a spherical shape,
they have a hydrodynamic diameter ranging from 0.6 to 2.0 nm, and preferably of less than 1.0 nm,
they have a metal core diameter ranging from 0.5 to 1.5 nm, and preferably of less than 1.0 nm,
they exhibit a stability duration ranging from 5 to 20 weeks when the nanoclusters are in liquid form and are stored at a temperature of 4° C.,
they exhibit a stability duration of at least 12 months, and preferably from 12 to 18 months,
   when the nanoclusters are in dry form and are stored at a temperature of 4° C. and under nitrogen,
they exhibit spectrophotometric properties, with a shoulder in the UV-Visible spectrum at 300±15 nm and a fluorescence spectrum with excitation wavelengths of 364±15 nm and emission wavelengths of 415±15 nm,
   it being possible to refer to said iron nanoclusters by using the formula “FeNC@HisAcAsc”.

The nanoclusters of the invention may, however, be referred to hereinafter either as “nanoclusters”, “iron nanoclusters”, “FeNC nanoclusters”, “FeNC”, “NC—Fe”, (“FeNC” or “NC-Fe” meaning “iron nanoclusters”), “FeNC@HisAcAsc nanoclusters”, or “FeNC@HisAcAsc”.

The formula “FeNC@HisAcAsc” is the most explicit, since it describes that the iron nanocluster comprises, on its surface, a layer comprising histidine as well as acetate ions and ascorbate ions.

The invention also relates to a method of preparing said iron nanoclusters, which comprises the following steps:
reaction of iron (II) acetate with histidine in order to obtain a mixture of iron acetate and histidine, the molar ratio of histidine/iron (II) acetate being greater than or equal to 8, preferably ranging from 8 to 200, and more preferably ranging from 80 to 200,
reaction of the mixture of iron acetate and histidine with ascorbic acid in order to obtain a mixture of iron acetate, histidine, and ascorbic acid, the molar ratio of ascorbic acid/iron (II) acetate being greater than or equal to 12, preferably ranging from 12 to 700, and more preferably ranging from 130 to 700,
collecting the iron nanoclusters covered on their surface with a mixed layer comprising histidine, acetate ions, and ascorbate ions.

The invention also relates to iron nanoclusters for use:
in the prevention and/or treatment of diseases causing iron deficiency (such as iron deficiency anemia),
in combating iron deficiencies.

Finally, the invention also relates to a composition comprising the iron nanoclusters of the invention, said composition being a medication, a dietary supplement, or a food composition.

The composition of the invention is further characterized in that it is in a form suitable for oral administration.

### BRIEF DESCRIPTION OF DRAWINGS

Other features, details, and advantages will become apparent upon reading the detailed description below and analyzing the attached figures.

FIG. 1 is a schematic representation of an iron nanocluster “FeNC@HisAcAsc” of the invention, consisting of an iron metal core surrounded by a mixed corona comprising histidine, acetate ions, and ascorbate ions.

FIG. 2 is a high-performance liquid chromatography analysis (reversed-phase separation) of the iron nanoclusters, showing the presence of acetate ions on the surface of the iron metal core.

The chromatogram was obtained on previously purified fractions (size exclusion chromatography) of iron nanoclusters. A chromatogram of a reference solution of sodium acetate was also performed.

FIG. 3 is a high-performance liquid chromatography analysis (reversed-phase separation) of the iron nanoclusters, showing the presence of histidine and ascorbate ions on the surface of the metal iron core.

The chromatogram was obtained on previously purified fractions (size exclusion chromatography) of iron nanoclusters. A chromatogram of a reference solution of histidine and ascorbic acid was also performed.

FIG. 4 illustrates the hydrodynamic diameter (in nanometers) of iron nanoclusters evaluated by dynamic light scattering.

FIG. 5 illustrates the hydrodynamic diameter (in nanometers) of iron nanoclusters evaluated by Taylor dispersion analysis.

FIG. 6 is a UV-Visible spectrum of the iron nanoclusters.

FIG. 7 is a fluorescence spectrum of the iron nanoclusters.

FIG. 8 illustrates the results of the viability assay (MTT) in HepG2 cells (human liver cancer cells) that have proliferated in the presence of iron, said iron being in the form of iron (III) nitrate (control, standard) (represented by FeNO₃) or in the form of iron nanoclusters of the invention at varying concentrations (represented by NC—Fe 1×; NC—Fe ½, NC—Fe ¼, NC—Fe ⅛, NC—Fe 1/16, and NC—Fe 1/32).

The “Negative” corresponds to the “IMDM” culture medium not supplemented with iron.

The histogram with horizontal hatching on the far left of each group of histograms corresponds to day D1+3 of the cell viability assay. The histogram immediately next to it corresponds to day D1+5, and the one after corresponds to day D1+7. The black histogram on the far right of each group of histograms corresponds to day D1+10.

### DETAILED DESCRIPTION

### Iron Nanoclusters

The present invention therefore relates to iron nanoclusters characterized in that they:
are covered on their surface with a mixed layer comprising histidine (His), acetate ions (Ac), and ascorbate ions (Asc),
have a spherical shape,
have a hydrodynamic diameter ranging from 0.6 to 2.0 nm, and preferably of less than 1.0 nm,
have a metal core diameter ranging from 0.5 to 1.5 nm, and preferably of less than 1.0 nm,
exhibit a stability duration ranging from 5 to 20 weeks when the nanoclusters are in liquid form and are stored at a temperature of 4° C.,
exhibit a stability duration of at least 12 months, and preferably from 12 to 18 months, when the nanoclusters are in dry form and are stored at a temperature of 4° C. and under nitrogen,
exhibit spectrophotometric properties, with a shoulder in the UV-Visible spectrum at 300±15 nm and a fluorescence spectrum with excitation wavelengths of 364±15 nm and emission wavelengths of 415±15 nm,
   it being possible to refer to said iron nanoclusters by using the formula “FeNC@HisAcAsc”.

The iron nanoclusters that are the object of the invention are metal nanoclusters. A metal nanocluster consists of the combination of several tens of atoms of a metallic element (in this case the iron of the invention) with a metal core diameter that is less than or equal to 2.0 nanometers (nm).

The nanoclusters of the invention are composed of an iron metal core covered/wrapped/surrounded by a mixed layer/corona comprising histidine, acetate ions, and ascorbate ions.

The terms “corona” and “layer” may be used interchangeably throughout this application.

The term “mixed” is used to indicate that the corona or layer surrounding the iron core comprises histidine, acetate ions, and ascorbate ions.

Similarly, the verbs “cover/wrap/surround” may be used interchangeably to indicate that the iron core comprises, over its entire surface, a layer/corona of histidine, acetate ions, and ascorbate ions.

The iron nanocluster as a whole has a spherical shape.

The formula “FeNC@HisAcAsc” within the meaning of the invention designates a nanocluster composed of an iron metal core coated with said mixed layer of histidine, acetate ions, and ascorbate ions. The iron nanoclusters of the invention thus advantageously comprise three ligands on the surface of the iron core, namely histidine, acetate ions, and ascorbate ions. These three ligands are bound to the iron metal core by coordination bonds.

In particular, the mixed corona comprising histidine, acetate ions, and ascorbate ions imparts very high stability and low reactivity to the nanoclusters of the invention.

“Low reactivity” means low degradation, in particular as related to oxidation (due to atmospheric oxygen for example).

The stability of the nanoclusters of the invention means that the structure and properties of the nanoclusters are maintained over time at a storage temperature of 4° C. A maintained structure means in particular that the composition of the nanocluster (metal core surrounded by the mixed layer/corona as defined above), its shape, and its diameter (metal core and hydrodynamic diameters) are preserved over time.

“Liquid form” of the iron nanoclusters refers to a solution or liquid mixture of iron nanoclusters. The aforementioned stability of 5 to 20 weeks applies to iron nanoclusters in liquid form when stored at a storage temperature of 4° C.

“Dry form” refers to a solid form that can be ground into powder if necessary. The aforementioned stability of 12 to 18 months applies to iron nanoclusters in dry form when stored at a storage temperature of 4° C. and under nitrogen.

Depending on their form (liquid or solid), their stability duration will therefore vary.

The nanoclusters of the invention exhibit spectrophotometric properties, particularly fluorescence properties, that are characteristic of this scale, namely a metal core diameter that is less than or equal to 2 nm, which is intermediate between a molecule and a nanoparticle.

As its name suggests, the metal core diameter, or metal diameter, refers to the diameter formed solely by the iron metal.

The hydrodynamic diameter includes the diameter of the iron core plus its layer/corona of histidine, acetate ions, and ascorbate ions. The hydrodynamic diameter therefore refers to the diameter of the entire iron nanocluster.

According to one embodiment of the invention, the iron nanoclusters have a metal core diameter and a hydrodynamic diameter which are more or less equivalent, and preferably less than 1.0 nm.

However, the metal core diameter will, of course, always be less than the hydrodynamic diameter.

The metal core diameter is evaluated by transmission electron microscopy, while the hydrodynamic diameter is evaluated by dynamic light scattering and/or Taylor dispersion analysis.

According to one advantageous embodiment of the invention, the iron nanoclusters are in liquid form or dry form.

The dry form of the nanoclusters is advantageous in particular in that it enables easy storage, preservation, and transport.

According to yet another advantageous embodiment, the iron nanoclusters of the invention are characterized in that they exhibit at least one of the following characteristics:
they are able to cross the intestinal barrier,
they exhibit good bioavailability,
they are biocompatible,
they are biodegradable,
they are able to be freeze-dried,
they are non-toxic to the human body,
they do not accumulate in organs such as the liver, spleen, kidneys, or lungs.

According to one advantageous embodiment, the iron nanoclusters of the invention exhibit all of the characteristics described above.

The fact that the nanoclusters of the invention are not sequestered in said organs is due in particular to their small size (hydrodynamic diameter that is less than or equal to 2.0 nm, and preferably less than 1.0 nm). The size of the nanoclusters of the invention allows for longer circulation in the blood, compared to larger compounds.

More specifically, the small size of the nanoclusters allows them to cross membranes (particularly gastrointestinal) without passing through physiological absorption systems via a persorption phenomenon (spontaneous passage through the pores of a physiological system). This persorption phenomenon is where the risk of toxicity from the nanoclusters originates, but it becomes a therapeutic modality if the quantitative aspect of nanocluster ingestion is controlled.

The nanoclusters of the invention possess surface properties that allow them to cross the intestinal barrier, which represents a significant advantage over oral iron preparations which are often unable to cross the intestinal barrier.

“Good bioavailability” means that orally administered iron nanoclusters reach the systemic circulation and are well distributed to target organs.

“Biocompatibility” means that the iron nanoclusters are well accepted by the body's various organs without being toxic to these organs.

The fact that the nanoclusters are biodegradable means that their degradation releases substances that are metabolized or eliminated by the body without problem (iron, histidine, acetate, and ascorbate).

According to one advantageous embodiment of the invention, it is possible to freeze-dry the nanoclusters.

It is possible to freeze-dry them because they are completely stable. Freeze-drying thus makes it easy to store, preserve, and transport the nanoclusters. The stability of the nanoclusters is as defined above.

The advantageous properties of the nanoclusters of the invention are due in particular to the unique combination of their components, namely iron, histidine, acetate ions, and ascorbate.

To the inventors' knowledge, iron nanoclusters comprising a mixed corona/layer of histidine, acetate ions, and ascorbate ions surrounding an iron metal core and exhibiting the advantageous properties described above have never been described before.

### Method of Preparing Iron Nanoclusters

The present invention also relates to a method of preparing iron nanoclusters as defined above, characterized in that it comprises the following steps:
reaction of iron (II) acetate with histidine in order to obtain a mixture of iron acetate and histidine, the molar ratio of histidine/iron (II) acetate being greater than or equal to 8, preferably ranging from 8 to 200, and more preferably ranging from 80 to 200.
reaction of the mixture of iron acetate and histidine with ascorbic acid in order to obtain a mixture of iron acetate, histidine, and ascorbic acid, the molar ratio of ascorbic acid/iron (II) acetate being greater than or equal to 12, preferably ranging from 12 to 700, and more preferably ranging from 130 to 200,
collecting the iron nanoclusters.

The molar ratios defined above, respectively between histidine and iron acetate, and between ascorbic acid and iron acetate, are important in that they allow the histidine ligands, acetate ions, and ascorbate ions to bind to the iron metal core. This results in nanoclusters comprising three ligands on the surface of the iron core, these three ligands being bound to the surface of the iron core by coordination bonds.

Ascorbic acid is a reducing agent. The reaction of the mixture of iron acetate and histidine with ascorbic acid is more particularly a reduction reaction of the mixture of iron acetate and histidine with ascorbic acid. Ascorbic acid makes it possible, in particular, to obtain iron nanoclusters that are without any toxicity.

The present invention results in particular from the inventors' unexpected discovery that the original combination of reagents used, namely iron acetate, histidine, and ascorbic acid, and in the proportions as defined above, allows obtaining iron nanoclusters with particularly advantageous properties.

The excellent stability of the nanoclusters of the invention is one example.

According to one embodiment of the invention, the iron nanoclusters may be prepared more particularly according to the “solution-phase” protocol or according to the “solid-phase” protocol. Each of these two synthesis routes is in accordance with the method described above.

### 1/Solution-Phase Protocol

According to one advantageous embodiment of the invention, the method of preparation as defined above is more particularly characterized in that it is carried out under inert gas and in that:
the iron acetate is in solution form and the histidine is in powder form,
a solution of iron acetate and histidine is prepared by adding histidine to the iron acetate solution,
the solution of iron acetate and histidine is adjusted to a pH value ranging from 11 to 13, and preferably 12,
the ascorbic acid is in powder form,
a solution of iron acetate, histidine, and ascorbic acid is prepared by adding ascorbic acid to the solution of iron acetate and histidine for which the pH has been adjusted to the aforementioned values,
the solution of iron acetate, histidine, and ascorbic acid is stirred for 2 to 6 hours, and preferably 4 hours, at a temperature ranging from 35° C. to 45° C., and preferably 40° C.,
a solution comprising iron nanoclusters is obtained at the end of the previous stirring step,
the solution comprising iron nanoclusters is optionally dialyzed to obtain a purified solution of iron nanoclusters,
the solution comprising iron nanoclusters, optionally dialyzed, is optionally freeze-dried to obtain a dry form of iron nanoclusters.

The solution comprising iron nanoclusters, optionally dialyzed, has a stability duration ranging from 5 to 20 weeks at a storage temperature of 4° C.

Dialysis allows removing anything not bound to the iron metal core, such as excess histidine or ascorbic acid, or any residual iron present in the nanocluster solution. The layer comprising histidine, acetate ions, and ascorbate ions is bound to the iron metal core by coordination bonds.

The dry form of the iron nanoclusters, obtained after freeze-drying, has a stability duration of at least 12 months, and preferably from 12 to 18 months, at a storage temperature of 4° C. and under nitrogen.

The dry form of the iron nanoclusters may be reconstituted at any time by mixing into a reconstitution solvent such as purified water. “Reconstitute/Reconstitution” refers to the simple process of mixing the dry form or lyophilizate with a solvent.

Analysis of the solution of iron nanoclusters that is obtained after reconstitution of the dry form shows that the iron nanoclusters exhibit all of the properties defined above and are therefore exactly the same as those obtained directly from their method of preparation.

The solution of iron nanoclusters obtained after reconstitution of the dry form exhibits a stability duration ranging from 5 to 12 weeks at a storage temperature of 4° C., preferably under nitrogen.

The method of preparation as defined above is further characterized in that it further comprises at least one characteristic selected from the following:
the inert gas is nitrogen,
the iron acetate solution is prepared by adding iron acetate to filtered ultrapure water,
the iron acetate solution has a concentration ranging from 0.5 to 5.0 mM,
the histidine concentration is higher than the concentration of the iron acetate solution,
the pH of the solution of iron acetate and histidine is adjusted using sodium hydroxide,
the ascorbic acid concentration is equal to the histidine concentration,
the solution comprising iron nanoclusters, optionally dialyzed, has an iron concentration ranging from 14 to 112 μg/mL,
the solution comprising iron nanoclusters, optionally dialyzed, is freeze-dried to obtain a dry form of iron nanoclusters.

According to one advantageous embodiment, the method of the invention exhibits all of the characteristics described above.

### 2/Solid-Phase Protocol

According to another advantageous embodiment of the invention, the method of preparing iron nanoclusters as defined above is more particularly characterized in that:
the iron acetate is in powder form and the histidine is in powder form,
a powder mixture of iron acetate and histidine is obtained by mixing together each of the iron acetate and histidine powders,
the powder mixture of iron acetate and histidine is ground until a powder mixture of homogeneous color is obtained,
the homogeneous powder mixture of iron acetate and histidine is placed in a reactor,
the ascorbic acid is in powder form,
the ascorbic acid is added to the reactor comprising the homogeneous powder mixture of iron acetate and histidine,
the thus obtained powder mixture of iron acetate, histidine, and ascorbic acid is stirred, then water is added dropwise into the reactor, said water being filtered ultrapure water.
the reactor is placed under inert gas and protected from light.
the mixture of iron acetate, histidine, ascorbic acid, and water is left to stir in the reactor for 16 to 36 hours, and preferably 24 hours.
a liquid mixture comprising iron nanoclusters is obtained at the end of the previous stirring step.
the liquid mixture comprising iron nanoclusters is optionally dialyzed to obtain a purified liquid mixture of iron nanoclusters.
the liquid mixture comprising iron nanoclusters, optionally dialyzed, is optionally freeze-dried to obtain a dry form of iron nanoclusters.

The method of preparation as defined above is further characterized in that it further comprises at least one characteristic selected from the following:
the histidine concentration is greater than the iron acetate concentration,
the ascorbic acid concentration is equal to the histidine concentration,
the water added to the reactor is filtered ultrapure water,
the inert gas is nitrogen,
the liquid mixture comprising the iron nanoclusters, optionally dialyzed, has an iron concentration ranging from 1500 to 15000 μg/mL,
the liquid mixture comprising iron nanoclusters, optionally dialyzed, is freeze-dried to obtain a dry form of iron nanoclusters.

According to another embodiment of the invention, the dry form of the iron nanoclusters obtained after freeze-drying (according to the “solution-phase” protocol or the “solid phase” protocol) is stored under nitrogen, preferably in vials, and preferably at 4° C. Under such conditions, the iron nanocluster powder may be stored for a period of at least 12 months, and preferably from 12 to 18 months, with no change in the stability of the iron nanoclusters.

Reconstitution of the nanocluster powder at the end of this period shows that the nanoclusters are the same as those obtained directly after their preparation (according to the “solution-phase” protocol or the “solid phase” protocol). Indeed, the iron nanoclusters exhibit all of the properties defined above.

### Usage of Iron Nanoclusters

The invention also relates to iron nanoclusters as defined above or obtained according to the methods as defined above, for use as medication.

More particularly, the invention relates to iron nanoclusters as defined above or obtained according to the methods as defined above, for use in the prevention and/or treatment of diseases causing iron deficiency.

An example of a disease causing iron deficiency is iron deficiency anemia.

The invention also relates to iron nanoclusters as defined above or obtained according to the methods as defined above, for use in combating iron deficiency.

In the present application, iron deficiency refers to iron deficiency in the broad sense, namely iron deficiency with or without iron deficiency anemia.

Another object of the invention is a composition characterized in that it comprises iron nanoclusters as defined above or obtained according to the methods as defined above.

The composition of the invention may be a medication, a dietary supplement, or a food composition.

The amount of iron per composition can determine whether it is a dietary supplement or a medication. Thus, a dietary supplement should contain less iron than a medication.

An example of a food composition includes, for example, infant formulas that are supplemented with iron, and more particularly with the iron nanoclusters of the invention.

According to one advantageous embodiment of the invention, the composition is in a form suitable for oral administration.

The iron nanoclusters advantageously may be administered orally because they are able to pass through the intestinal barrier without difficulty, in particular due to their small size.

Use of the iron nanoclusters of the invention advantageously eliminates the need for intravenous administration.

According to another advantageous embodiment, the composition of the invention comprising the iron nanoclusters comprises a lower amount of iron than the amount of iron usually present in a conventional oral preparation, whether a medication or a dietary supplement.

Advantageously, the composition of the invention does not exhibit the disadvantages that may be encountered with a conventional oral preparation, whether a medication or a dietary supplement.

### EXAMPLES

The following examples illustrate the invention, but do not limit it in any way.

### Example 1

### Preparation of Iron Nanoclusters

This example describes the two possible synthesis routes for preparing the iron nanoclusters of the invention, namely the “solution-phase protocol” and the “solid-phase protocol.”

### 1/Solution-Phase Protocol

### Reagents Used:

Iron (II) acetate [Fe(CH₃COO)₂], M=171.83 g/mol (Sigma Aldrich, Cas 3094-87-9);
L(−)-Histidine, M=155.15 g/mol (Merck, Cas 71-00-1);
Ascorbic acid, M=176.12 g/mol (Sigma Aldrich, Cas 50-81-7);
1M NaOH solution, M=40.00 g/mol (VWR, Cas 1310-73-2).

### Precautions

The synthesis is carried out under inert gas (nitrogen). The glassware is washed with aqua regia (1 volume of 65% nitric acid to 2 volumes of 37% hydrochloric acid).

Ultrapure water is used and filtered through a 0.2 μm pore size filter.

The iron acetate is in powder form and stored under nitrogen. Once the iron is weighed, the remaining stock must be stored under nitrogen once again.

Since the iron nanoclusters are intended for in vivo study, it is necessary to work under a clean fume hood and to clean all the equipment used with 70% v/v ethanol.

### Preparation of a 2.5 mM Iron Acetate Stock Solution

42.9 mg of iron acetate is placed in a 100 mL volumetric flask. Filtered ultrapure water is added up to the flask's fill line. An iron acetate solution with a concentration of 2.5 mM is obtained.

After complete dissolution, the iron acetate solution is transferred into a suitable container. This solution can be stored for one month in a refrigerator at 4° C.

Synthesis of Nanoclusters Stabilized with Histidine

500 μL of the iron acetate stock solution prepared in the previous step is added to a round-necked flask capable of holding up to 50 mL of solution. Then, 4500 μL of filtered ultrapure water is added to the flask. The resulting iron acetate solution is referred to as 1×.

The 1×iron acetate solution is stirred at 130 rpm using a multi-plate shaker. 39 mg of histidine is added to the iron acetate solution. The iron acetate and histidine solution is stirred for 15 minutes.

The solution takes on a slightly red tint. After 15 minutes of stirring, the pH of the iron acetate and histidine solution is adjusted to 12 with 10 drops of 1M NaOH. 139 mg of ascorbic acid is added to the reaction mixture. 2 minutes is allowed for the ascorbic acid to dissolve completely.

The flask (reactor) is placed in a water bath at 40° C. with stirring (speed set to 6) for 4 hours.

At the end of the synthesis, the resulting solution of nanoclusters is colorless. The resulting solution of iron nanoclusters is referred to as 1× and has an iron concentration of 14 μg/mL. It is stored in a cool location at 4° C.

The synthesis yield is 100%: there is no residual iron (iron element) in the nanocluster solution.

The iron nanocluster solutions may be freeze-dried.

Iron acetate solutions with concentrations ranging from 1× to 8×are prepared in order to obtain 1× to 8×solutions of iron nanoclusters which have an iron concentration ranging from 14 to 112 μg/mL.

For reference, a 2×iron acetate solution is prepared by placing 1000 μL of iron acetate stock solution in a flask and topping up to 5000 μL with filtered ultrapure water. A 4×iron acetate solution is prepared by placing 2000 μL of iron acetate stock solution in a flask and topping up to 5000 μL with filtered ultrapure water, etc.

The resulting 1× to 8×solutions of iron nanoclusters are stored in a cool location at 4° C.

### Dialysis of Iron Nanoclusters

The 1×solution of iron nanoclusters that is obtained in the previous step is purified by dialysis. A dialysis device is prepared (X12 Float-a-lyzer G2 CE MWCO 100-500 D, Reference 1511160), and a 150 ml beaker is filled with 100 mL of filtered ultrapure water. The dialysis device is filled with filtered ultrapure water using a Pasteur pipette. The dialysis device is placed in the beaker while stirring (130 rpm). The device is left to hydrate and wash for 1 hour.

The water is then replaced with a new volume of 100 mL of filtered ultrapure water. The dialysis device is emptied using a Pasteur pipette and then filled with the 1×solution of iron nanoclusters, which is left to stir overnight (for 12 hours) at a temperature between 2 and 6° C. The resulting dialyzed 1×solution of iron nanoclusters is transferred into a suitable container and stored at a temperature of 4° C.

Dialysis does not affect the iron concentration of the nanoclusters. Thus, the iron concentrations of the dialyzed solutions of iron nanoclusters are identical to those of non-dialyzed solutions.

The solutions of iron nanoclusters, if dialyzed, may be freeze-dried.

The iron concentrations of the dialyzed solutions of iron nanoclusters are identical to those of non-dialyzed solutions, and range from 14 to 112 μg/mL for iron acetate solutions with concentrations ranging from 1× to 8×.

### 2/Solid-Phase Protocol

### Reagents Used and Precautions

The iron (II) acetate, L(−)-Histidine, and ascorbic acid are the same as those used in the solution-phase protocol. Sodium hydroxide is not required in the solid-phase protocol.

The same precautions are required as for the solution-phase protocol.

### Synthesis of Iron Nanoclusters Stabilized With Histidine

A 23 mg quantity of iron acetate is weighed and then placed inside an agate mortar. 1.7 g of histidine is then weighed. One part histidine powder to one part iron acetate powder is added, taking care to grind the powders thoroughly with a pestle until a mixture of uniform color and appearance is obtained. This operation is repeated until all the histidine has been used.

The final mixture of the two powders should have a red color and the powder should be homogeneous. The mixture of the two powders is then transferred to a 50 mL single-necked flask (NS 19/26 ground neck) using a spatula. 3 g of ascorbic acid is weighed and transferred into the flask. An olive-shaped magnetic stirrer is placed at the bottom of the flask. 5 mL of ultrapure water is filtered using a 5 mL plastic syringe and is added dropwise into the reactor. A liquid mixture is obtained.

The reactor is closed using a flip-top skirt cap (19.4 mm diameter) and is placed under nitrogen without creating excess pressure, using a latex balloon. The reactor is wrapped in aluminum foil and then stirred (200 rpm) for the duration of the reaction. A 24-hour wait is required for the reaction to complete. At the end of the reaction, the resulting product, which is in liquid form, is red in color.

The resulting liquid comprising the iron nanoclusters has an iron concentration of 1500 μg/mL and is referred to as 100×.

The iron concentration of the nanoclusters is, of course, dependent on the amount of iron acetate used at the start of the method of the invention.

The operations described above are repeated, to obtain iron concentrations ranging from 1500 (100×) to 15,000 μg/mL (1000×) for quantities of iron acetate at the start of the method of the invention (solid-phase protocol) which range from 23 to 230 mg.

The resulting liquid comprising the iron nanoclusters is then transferred into a suitable plastic container (the final volume is not 5 mL but slightly larger, approximately 8.5 mL). The liquid comprising the iron nanoclusters is either stored at 4° C. or transferred to a freeze-dryer.

The freeze-drying is carried out in 1 mL portions, with no addition of additional reagents. After freeze-drying, the contents of the vial (which includes the iron nanoclusters in dry form) are placed under nitrogen and stored at 4° C.

The dry form of the iron nanoclusters may be reconstituted at any time in 1 mL of purified water. The solution of iron nanoclusters reconstituted in this manner is stored at 4° C., preferably under nitrogen.

### Example 2

### Characterization of the Iron Nanoclusters

The iron nanoclusters obtained in Example 1, whether via the solution-phase or solid-phase protocol, are characterized in terms of their structure, size, spectrophotometric properties, and stability.

### Structure of the Iron Nanoclusters

The iron nanoclusters of the invention have, more particularly, a spherical shape. They are composed of an iron metal core covered with a mixed corona comprising histidine, acetate ions, and ascorbate ions. FIG. 1 is a schematic representation of an iron nanocluster of the invention, which may also be designated by the formula “FeNC@HisAcAsc”.

The presence of acetate ions on the surface of the iron nanoclusters was demonstrated by high-performance liquid chromatography (HPLC), more specifically by reversed-phase chromatography.

The iron nanoclusters in the 1×dialyzed solution as obtained in Example 1, see section 1/“Solution-phase protocol,” are destroyed (total dissolution and return of the nanocluster structure to its various component elements) by a chemical process, namely dissolution in a concentrated acid (HCl) then in a concentrated base (NaOH), then are analyzed by HPLC in comparison to a sodium acetate control.

The results obtained are illustrated in the chromatogram in FIG. 2. The peak at 3.6 min associated with acetate ions (control, see lower plot) is found in the iron nanoclusters (see upper plot), thus demonstrating the presence of acetate ions on the surface of the metal core. The presence of histidine and ascorbate ions on the surface of the iron nanoclusters was also demonstrated by high-performance liquid chromatography, more particularly after purification of the nanoclusters in solution by size exclusion chromatography, in comparison to a histidine and ascorbic acid control.

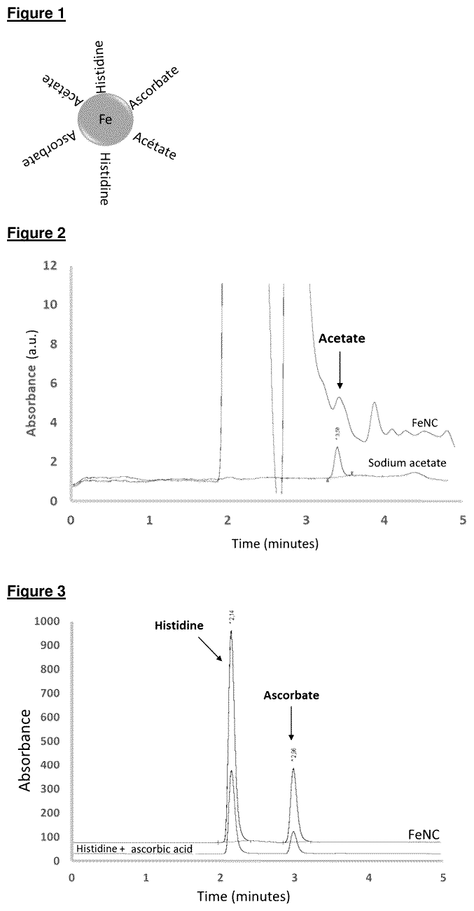

The results obtained are illustrated in the chromatogram in FIG. 3.

The peak at 2.1 min associated with histidine (control, see lower plot) is found in the iron nanoclusters (see upper plot), thus demonstrating the presence of histidine on the surface of the metal core.

The peak at 3.0 min associated with ascorbate ions (control, see lower plot) is found in the iron nanoclusters (see upper plot), thus demonstrating the presence of ascorbate ions on the surface of the metal core.

### Size of the Iron Nanoclusters

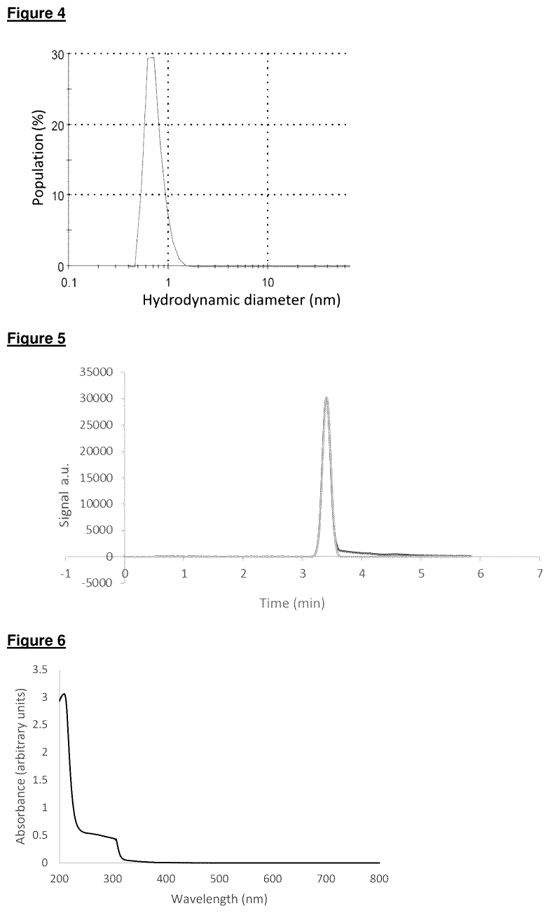

The hydrodynamic diameter (Dh) of the iron nanoclusters was evaluated by dynamic light scattering (FIG. 4) (173° angle, 530 nm laser, 25° C. temperature in a Malvern Nanosizer) and by Taylor dispersion analysis (FIG. 5).

The method using dynamic light scattering analysis consists of analyzing the Brownian motion of particles and modeling this using the Stokes-Einstein equation.

The method using Taylor dispersion analysis involves injecting a plug of solute into an open capillary tube (50 μm) where it disperses under the influence of a hydrodynamic flow (positive pressure 1 psi, parabolic velocity profile). The principle for determining the hydrodynamic radius is based on the Taylor-Aris relationship, which establishes the link between the spread of the solute peak (modeling a Gaussian) and the molecular diffusion coefficient.

The metal core diameter of the iron nanoclusters was evaluated using transmission electron microscopy (deposition on nickel grids, observations under beams operating at 200 kV (LaB6 cathode) Philips CM 200).

The diameters (hydrodynamic and metal core) of the iron nanoclusters were evaluated immediately after their synthesis, for both the solution-phase and solid-phase protocols.

For the solution-phase protocol, the hydrodynamic and metal core diameters were evaluated in the 1×solution of iron nanoclusters, non-dialyzed and not freeze-dried, which has an iron concentration of 14 μg/mL.

For the solid-phase protocol, the hydrodynamic and metal core diameters were evaluated in the freeze-dried samples obtained from the 100×liquid of iron nanoclusters, non-dialyzed, which has an iron concentration of 1500 μg/mL.

The average hydrodynamic diameter of the iron nanoclusters, as with the metal core diameter, is less than 1.0 nm. More specifically, FIGS. 4 and 5 show that, in dynamic light scattering (FIG. 4) and Taylor dispersion (FIG. 5), the hydrodynamic diameter of the iron nanoclusters is 0.69±0.06 nm.

### Spectrophotometric Properties

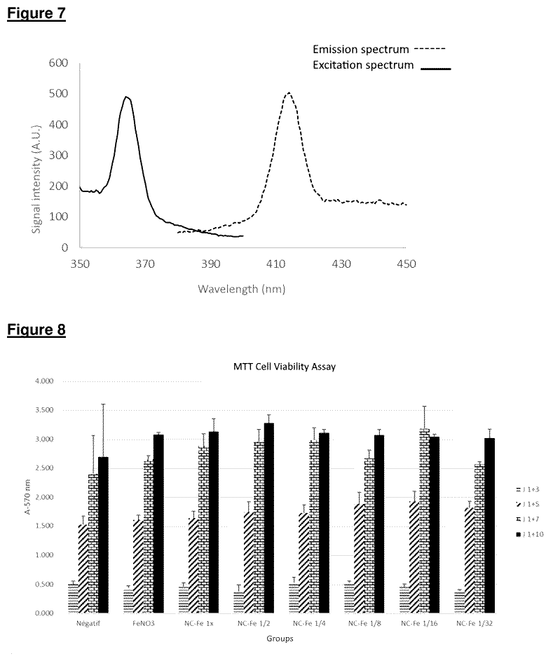

The spectrophotometric properties of the iron nanoclusters were evaluated by UV-Visible spectroscopy (FIG. 6) and fluorescence spectroscopy (FIG. 7), immediately after their synthesis.

The UV-vis spectrum shows a shoulder at 300±15 nm (FIG. 6), confirming the existence of the nanoclusters.

The iron nanoclusters exhibit fluorescence with an excitation wavelength of 364±15 nm and an emission wavelength of 415±15 nm (FIG. 7), also confirming the existence of the nanoclusters.

The iron nanoclusters of the invention exhibit optical properties, particularly fluorescence, which are characteristic of this intermediate scale between molecule and nanoparticle.

### Stability of the Iron Nanoclusters

The stability of the iron nanoclusters was assessed by measuring their hydrodynamic diameter using dynamic light scattering (173° angle, 530 nm laser, 25° C. temperature in a Malvern Nanosizer).

The analyses were performed on iron nanoclusters obtained using both the solution-phase protocol and the solid-phase protocol.

For the solution-phase protocol, the analyses were performed on 1×solutions of iron nanoclusters (iron concentration of 14 μg/mL). Stability was assessed slightly more than 5 weeks after synthesis.

It was found that the hydrodynamic diameter of the iron nanoclusters remained at 0.69±0.06 nm more than 5 weeks after synthesis, demonstrating their excellent stability.

For the solid-phase protocol, the analyses were performed on:
freeze-dried samples obtained from the 100×liquid of iron nanoclusters, non-dialyzed (iron concentration of 1500 μg/mL),
solutions reconstituted after freeze-drying said samples, to the same volume as was freeze-dried.

The hydrodynamic diameter of the iron nanoclusters in the freeze-dried samples was 0.70 nm after more than 5 weeks of storage under nitrogen.

After reconstitution of the samples to the same volume as was freeze-dried, the hydrodynamic diameter of the iron nanoclusters in the reconstituted samples was 0.76 nm, further demonstrating their excellent stability.

### Example 3

### Assessing the Toxicity of the Iron Nanoclusters

This example describes the results of the viability assay (MTT assay) on HepG2 cells (human liver cancer cells) that have proliferated in the presence of iron, said iron being in the form of iron (III) nitrate (control, standard) or in the form of the iron nanoclusters of the invention at varying concentrations.

### Iron

The iron (III) nitrate (or ferric nitrate) used as a control is the chemical compound with the semi-structural formula “Fe(NO₃)₃”, which more particularly is used in its nonahydrate form “Fe(NO₃)₃.9H₂O”.

A solution of iron nitrate nonahydrate is prepared, for a total volume of 50 mL at a concentration of 100 mg/L of water, which corresponds to a concentration of 14 μg/mL of iron. This solution is filtered under a PSM fume hood.

The 1×solution of iron nanoclusters, which contains an iron concentration of 14 μg/mL as prepared in Example 1 (section 1/Solution-phase protocol), is used in particular.

Serial dilution of the 1×solution of iron nanoclusters is performed to ½, ¼, ⅛, 1/16, and 1/32 (see prepared media 4 to 8 below).

### HepG2 Cells

HepG2 cells are a cell line derived from the liver tissue of a patient presenting hepatocellular carcinoma (HCC).

### MTT Assay

The MTT assay is a rapid colorimetric method for quantifying living cells within a sample. The reagent used is the tetrazolium salt “MTT” (“3-(4,5-dimethylthiazol-2-yl)-2,5-diphenyl tetrazolium bromide”). The tetrazolium ring it contains is reduced by the mitochondrial succinate dehydrogenase in active living cells, to formazan. This forms a purple precipitate in the mitochondrion.

The amount of precipitate formed is proportional to the number of living cells (but also to the metabolic activity of each cell). Therefore, after incubating the cells with MTT for a certain amount of time at 37° C. (approximately three hours), it is sufficient to dissolve the cells, their mitochondria, and therefore the purple formazan precipitates, in 100% DMSO (dimethyl sulfoxide).

A simple spectroscopic measurement of the optical density at 570 nm allows determining the relative amounts of living and metabolically active cells.

It is therefore necessary, if the assay is to be quantitative, to create a calibration curve for each assay. The reading is obtained at 570 nm by means of a spectrophotometer (see FIG. 8).

### HepG2 Cell Growth Conditions

HepG2 cells were grown in a complete culture medium (DMEM—Dulbecco's Modified Eagle's Medium) in 24-well plates for 24 hours. The medium was then changed to a selective culture medium (IMDM*) (see below) +/−iron nanoclusters of the invention versus iron nitrate nonahydrate Fe(NO₃)₃.9H₂O.

Fe³⁺ was reduced to Fe²⁺ and then passed through the plasma membrane via DMT1 (divalent metal transporter 1).

### Selective Culture Medium (IMDM*):

IMDM (Iscove's Modified Dulbecco's Medium),
10% FBS (fetal bovine serum) (dialyzed),
1% antibiotics (mixture of penicillin and streptomycin),
1% (1 mM) pyruvate.

### Eight Selective Media are Prepared:

Medium 1: IMDM*;
Medium 2: IMDM*+1% Fe(NO₃)₃.9H₂O (100 mg/L stock);
Medium 3: IMDM*+1% 1×solution of iron nanoclusters (i.e., 14 μg/mL equivalent iron);
Medium 4: 1:2 dilution of Medium 3 in IMDM*;
Medium 5: 1:2 dilution of Medium 4 in IMDM* (i.e., ¼ of Medium 3);
Medium 6: 1:2 dilution of Medium 5 in IMDM* (i.e., ⅛ of Medium 3);
Medium 7: 1:2 dilution of Medium 6 in IMDM* (i.e., 1/16 of Medium 3);
Medium 8: 1:2 dilution of Medium 7 in IMDM* to ½ (i.e., 1/32 of Medium 3).

### Assay Duration

One plate is prepared for each step (D1+3, D1+5, D1+7, D1+10). The overall assay duration is 11 days.

The steps are as follows:
Day 0: Seeding in p24 (triplicates), 4000 cells/cm², DMEM complete medium (500 μL/well);
Day 1: Change to IMDM selective medium, solution of iron nanoclusters versus Fe(NO₃)₃.9H₂O (0.1 mg/L);
Day 1+3: 1 plate stopped for MTT, medium changed for the rest;
Day 1+5: 1 plate stopped for MTT, medium changed for the rest;
Day 1+7: 1 plate stopped for MTT, medium changed for the rest;
Day 1+10: 1 plate stopped for MTT, end of test.

The obtained results are described in FIG. 8, where:
“Negative” corresponds to Medium 1,
“FeNO3” corresponds to Medium 2,
“NC—Fe 1×” corresponds to Medium 3,
“NC—Fe ½” corresponds to Medium 4,
“NC—Fe ¼” corresponds to Medium 5,
“NC—Fe ⅛” corresponds to Medium 6,
“NC—Fe 1/16” corresponds to Medium 7,
“NC—Fe 1/32” corresponds to Medium 8.

The MTT viability assays repeated on D+3, D+5, D+7, and D+10 indicate that treatment with the iron nanoclusters of the invention is not toxic to HepG2 cells treated with an equivalent dose of iron found in the standard culture media in the form of iron nitrate nonahydrate Fe(NO₃)₃.9H₂O at a concentration of 0.1 mg/L.

The negative control was treated with medium without added iron, but the presence of fetal bovine serum provided sufficient iron to allow some cell growth (therefore, the control is not completely negative).

The MTT was weighed, dissolved at 5 mg/mL in native IMDM medium, filtered at 0.2 μm under a PSM fume hood, and stored at +4° C. throughout the experimental protocol.

At each step of the MTT assay, the 5 mg/mL solution was diluted to 0.5 mg/mL in IMDM complete medium (IMDM+FBS+antibiotics+pyruvate), but without iron, and then incubated for 3 hours at 37° C.

After each change of medium, the spent medium was removed and frozen at −20° C. for subsequent transferrin and ferritin tests.

In conclusion, this assay demonstrates that the iron nanoclusters of the invention do not present any toxicity, regardless of their concentration. HepG2 cells cultured in a medium without iron and then with the addition of iron show excellent viability.

1-16. (canceled)

17. Iron nanoclusters comprising a surfaced covered with a mixed layer comprising histidine (His), acetate ions (Ac), and ascorbate ions (Asc), wherein the iron nanoclusters:
have a spherical shape,
have a hydrodynamic diameter ranging from 0.6 to 2.0 nm, and preferably of less than 1.0 nm,
have a metal core diameter ranging from 0.5 to 1.5 nm, and preferably of less than 1.0 nm,
exhibit a stability duration ranging from 5 to 20 weeks when the nanoclusters are in liquid form and are stored at a temperature of 4° C.,
exhibit a stability duration of at least 12 months, when the nanoclusters are in dry form and are stored at a temperature of 4° C. and under nitrogen,
exhibit spectrophotometric properties, with a shoulder on the UV-Visible spectrum at 300±15 nm and a fluorescence spectrum with excitation wavelengths of 364±15 nm and emission wavelengths of 415±15 nm,
wherein the iron nanoclusters are referred to as having the formula “FeNC@HisAcAsc”.

18. The iron nanoclusters according to claim 17, wherein the iron nanoclusters are in liquid form or dry form.

19. The iron nanoclusters according to claim 17, wherein the iron nanoclusters exhibit at least one of the following characteristics:
the iron nanoclusters are able to cross the intestinal barrier,
the iron nanoclusters exhibit good bioavailability,
the iron nanoclusters are biocompatible,
the iron nanoclusters are biodegradable,
the iron nanoclusters are able to be freeze-dried,
the iron nanoclusters are non-toxic to the human body, and
the iron nanoclusters do not accumulate in organs such as the liver, spleen, kidneys, or lungs.

20. A method of preparing iron nanoclusters according to claim 17, comprising the following steps:
reaction of iron (II) acetate with histidine in order to obtain a mixture of iron acetate and histidine, the molar ratio of histidine/iron (II) acetate being greater than or equal to 8,
reaction of the mixture of iron acetate and histidine with ascorbic acid in order to obtain a mixture of iron acetate, histidine, and ascorbic acid, the molar ratio of ascorbic acid/iron (II) acetate being greater than or equal to 12,
collecting the iron nanoclusters.

21. The method according to claim 20, wherein the method is carried out under inert gas and:
the iron acetate is in solution form and the histidine is in powder form,
a solution of iron acetate and histidine is prepared by adding histidine to the iron acetate solution,
the solution of iron acetate and histidine is adjusted to a pH value ranging from 11 to 13,
the ascorbic acid is in powder form,
a solution of iron acetate, histidine, and ascorbic acid is prepared by adding ascorbic acid to the solution of iron acetate and histidine for which the pH has been adjusted to the aforementioned values,
the solution of iron acetate, histidine, and ascorbic acid is stirred for 2 to 6 hours, and preferably 4 hours, at a temperature ranging from 35° C. to 45° C.,
a solution comprising iron nanoclusters is obtained at the end of the previous stirring step,
the solution comprising iron nanoclusters is optionally dialyzed to obtain a purified solution of iron nanoclusters, and
the solution comprising iron nanoclusters, optionally dialyzed, is optionally freeze-dried to obtain a dry form of iron nanoclusters.

22. The method according to claim 21, further comprising at least one characteristic selected from the following:
the inert gas is nitrogen,
the iron acetate solution is prepared by adding iron acetate to filtered ultrapure water,
the iron acetate solution has a concentration ranging from 0.5 to 5.0 mM,
the histidine concentration is higher than the concentration of the iron acetate solution,
the pH of the solution of iron acetate and histidine is adjusted using sodium hydroxide,
the ascorbic acid concentration is equal to the histidine concentration,
the solution comprising iron nanoclusters, optionally dialyzed, has an iron concentration ranging from 14 to 112 μg/mL, and
the solution comprising iron nanoclusters, optionally dialyzed, is freeze-dried to obtain a dry form of iron nanoclusters.

23. The method according to claim 20, wherein:
the iron acetate is in powder form and the histidine is in powder form,
a powder mixture of iron acetate and histidine is obtained by mixing together each of the iron acetate and histidine powders,
the powder mixture of iron acetate and histidine is ground until a powder mixture of homogeneous color is obtained,
the homogeneous powder mixture of iron acetate and histidine is placed in a reactor,
the ascorbic acid is in powder form,
the ascorbic acid is added to the reactor comprising the homogeneous powder mixture of iron acetate and histidine,
the thus obtained powder mixture of iron acetate, histidine, and ascorbic acid is stirred, then water is added dropwise into the reactor, said water being filtered ultrapure water,
the reactor is placed under inert gas and protected from light,
the mixture of iron acetate, histidine, ascorbic acid, and water is left to stir in the reactor for 16 to 36 hours,
a liquid mixture comprising iron nanoclusters is obtained at the end of the previous stirring step,
the liquid mixture comprising iron nanoclusters is optionally dialyzed to obtain a purified liquid mixture of iron nanoclusters, and
the liquid mixture comprising iron nanoclusters, optionally dialyzed, is optionally freeze-dried to obtain a dry form of iron nanoclusters.

24. The method according to claim 23, wherein the method comprises at least one characteristic selected from the following:
the histidine concentration is greater than the iron acetate concentration,
the ascorbic acid concentration is equal to the histidine concentration,
the water added to the reactor is filtered ultrapure water,
the inert gas is nitrogen,
the liquid mixture comprising iron nanoclusters, optionally dialyzed, has an iron concentration ranging from 1500 to 15000 μg/mL, and
the liquid mixture comprising iron nanoclusters, optionally dialyzed, is freeze-dried to obtain a dry form of iron nanoclusters.

25. The method according to claim 21, wherein the dry form of the iron nanoclusters is stored under nitrogen for a period of at least 12 months with no change in the stability of the iron nanoclusters.

26. A medication comprising the iron nanoclusters as defined in claim 17.

27. A method for preventing and/or treating diseases causing iron deficiency in a subject in need thereof, administering to said subject an effective amount of the iron nanoclusters as defined in claim 17.

28. The method according to claim 27, wherein the disease causing an iron deficiency is iron deficiency anemia.

29. A method for combating iron deficiencies in a subject in need thereof, administering to said subject an effective amount of the iron nanoclusters as defined in claim 17.

30. A composition comprising the iron nanoclusters as defined in claim 17.

31. The composition according to claim 30, wherein the composition is a medication, a dietary supplement, or a food composition.

32. The composition according to claim 30, wherein the composition is in a form suitable for oral administration.
